(19) United States
(12) Patent Application Publication
(10) Pub. No.: US 2002/0172663 A1
(43) Pub. Date: Nov. 21, 2002

(54) Localized myocardial injection method for treating ischemic myocardium

(76) Inventor: Maria Palasis, Wellesley, MA (US)

Correspondence Address: Hale and Dorr LLP, 300 Park Avenue, New York, NY, 10022, US

(21) Appl. No.: 10/057,409

(22) Filed: Jan. 23, 2002

Related U.S. Application Data
(60) Provisional application No. 60/263,468, filed on Jan. 23, 2001.

(51) Int. Cl. A61K 48/00; A61K 9/127

(52) U.S. Cl. 424/93.2; 424/450; 424/93.21

(57) ABSTRACT

This invention relates to a method of treating ischemic or diseased myocardium by injecting a therapeutic agent, such as a gene, protein, cell or drug, into normal myocardium, preferably adjacent to an ischemic zone in the heart of a subject. The method is useful for inducing angiogenesis and collateral blood vessel formation to improve cardiac function in subjects with ischemic heart disease. The method can also be used to promote tissue regeneration in such subjects.

### RELATED APPLICATIONS

This application claims the benefit of U.S. Provisional Application Serial No. 60/263,468, filed Jan. 23, 2001, the entire contents of which are incorporated herein.

### FIELD OF THE INVENTION

This invention relates to a method of treating ischemic or diseased myocardium by injecting a therapeutic agent, such as a gene, protein, cell or drug, into normal myocardium, preferably adjacent to an ischemic zone in the heart of a subject. The method is useful for inducing angiogenesis and collateral blood vessel formation to improve cardiac function in subjects with ischemic heart disease. The method can also be used to promote tissue regeneration in such subjects.

### BACKGROUND OF THE INVENTION

Cardiovascular diseases are generally characterized by an impaired supply of blood to the heart or other target organs. When the blood supply to the heart is compromised, cells respond by generating compounds that induce the growth of new vessels to increase the supply of blood to the heart. The process by which these new blood vessels, termed collateral blood vessels, are induced to grow out of the existing vasculature is termed angiogenesis, and the substances that are produced by cells to induce angiogenesis are termed angiogenic factors. As the body's natural angiogenic response is often inadequate, the use of exogenously supplied angiogenic factors is currently being explored as a means to treat cardiovascular disease.

Myocardial gene therapy can be used for the treatment of a number of cardiovascular diseases, including ischemic cardiomyopathies, congestive heart failure, and malignant arrhythmias (Nabel (1995) Circulation 91:541-548). Gene therapy to treat cardiac disease requires that gene therapy agents be delivered to the heart in a manner that will produce a favorable response. Intracoronary delivery of angiogenic growth factors and gene therapy vectors is possible, but this approach may result in dilution of the therapeutic agent due dispersal of the agent in the systemic circulation. Furthermore, such delivery methods may result in undesired side effects due to potential systemic distribution of such angiogenic agents, including vascularization of tumors and retinopathy. Intramyocardial injection provides a means to deliver angiogenic agents that avoids these pitfalls. Kornowski et al. (J. Am. Coll Cardiol. 35:1031-9, 2000) teaches the delivery of an angiogenic gene therapy vector directly to ischemic tissue using catheter-based and surgical techniques Post et al. (Card. and Vasc. Regeneration 2:106-113) discloses the transfection efficiency of transendocardial and direct epicardial injection of an angiogenic gene therapy vector.

It is currently unknown whether precise localization of intramyocardial injections is necessary. At present, most studies have targeted injections into the ischemic or diseased portion of the myocardium. However, injections could be placed, for example, in an ischemic area, in the zone bordering an ischemic area, or in normal myocardium.

In accordance with the present invention, it has surprisingly been found that a favorable functional response occurs when the angiogenic agent is injected into the normal myocardium, and more particularly into the normal myocardium adjacent to an ischemic zone.

### SUMMARY OF THE INVENTION

The present method of delivering a therapeutic agent to normal myocardium or normal myocardial tissue adjacent to a site of ischemia in an ischemic or diseased heart can be used to induce angiogenesis, to increase contractile function in the heart, to increase blood flow within the heart, to stimulate collateral vessel development in the heart, to promote tissue regeneration and to treat myocardial ischemia, particularly in a human patient.

In one aspect of the present invention, the invention provides a method for delivering a therapeutic agent to an ischemic or diseased heart by delivering a therapeutically-effective amount of the therapeutically effective agent to normal tissue in the ischemic or diseased heart. In accordance with the present invention, the therapeutic agent can be a transgene encoding an angiogenic protein or peptide that is delivered into the myocardium of the subject by intramyocardial injection of a gene therapy vector comprising that transgene. The vector is injected into normal tissue in the heart, and preferably into the non-ischemic or non-diseased myocardium adjacent to an ischemic or diseased zone in the heart. The gene therapy vector may be a plasmid or a viral vector, such as an adenoviral vector or recombinant adenoviral vector, or an adeno-associated vector or recombinant adeno-associated vector. The plasmid or viral vector may be delivered naked or in a liposome. Alternatively the therapeutic agent can be an angiogenic protein or peptide, a cell or cells, one or more drugs, an antisense DNA or RNA, or any other therapeutic agent useful to induce angiogenesis, increase contractile function in the heart, increase blood flow within the heart, stimulate collateral vessel development in the heart, promote tissue regeneration, improve exercise tolerance, or treat myocardial ischemia.

In another aspect of the present invention, the invention provides a method for stimulating collateral blood vessel formation in the myocardium, by intramyocardially delivering a sufficient amount of an angiogenic factor to normal tissue in an ischemic heart of a subject to stimulate collateral blood vessel formation. The angiogenic factor may be delivered, for example, by an adenovirus vector or an adeno-associated virus vector that comprises a coding sequence operatively linked to a promoter which induces expression of the coding sequence in a cardiac cell. The invention also provides methods for inducing collateral vessel formation in myocardium, inducing angiogenesis in myocardium, and improving contractile function of the heart. In these methods, an angiogenic factor, or cells capable of producing an angiogenic factor, is delivered intramyocardially to normal tissue of the diseased or damaged heart.

Still another aspect provides a method for promoting tissue regeneration in an ischemic or diseased heart of a subject by delivering a therapeutic agent, or cells capable of producing a therapeutic agent, to normal tissue in an ischemic or diseased heart of a subject in an amount sufficient to stimulate tissue regeneration in the heart. The therapeutic agent can be a protein or nucleic acid encoding, for example, a ligand for stem or progenitor cells, or any other agent which stimulates tissue regeneration.

In another aspect of the present invention, the invention provides a method for treating myocardial ischemia by delivering a therapeutic agent to normal myocardial tissue in an amount sufficient to ameliorate the symptoms of myocardial ischemia. In this aspect of the invention, amelioration of ischemia can include induction of angiogenesis, stimulation of collateral vessel development in the heart, tissue regeneration, improvement of contractile function in the heart, increased blood flow within the heart, increased tolerance to exercise, decreased angina pectoris, and relief of other symptoms and conditions associated with myocardial ischemia. The therapeutic agent can be delivered to multiple sites throughout the normal myocardium, or to a site or sites bordering the ischemic zone. Suitable therapeutic agents for use in this aspect of the invention include angiogenic proteins or peptides, transgenes encoding angiogenic proteins or peptides, a cell or cells, one or more drugs, antisense RNA or DNA, or other therapeutic agents.

### BRIEF DESCRIPTION OF THE DRAWINGS

FIG. 1 provides a schematic illustration of a porcine heart with placement of an ameroid constrictor for inducing chronic myocardial ischemia. The ischemic and non-ischemic areas are noted.

FIG. 2 shows anterior (top left), lateral (bottom left) and posterior (bottom right) views of a porcine heart and indicates the ischemic risk areas induced by an ameroid constrictor.

FIG. 3 is a bar graph depicting myocardial blood flow in pigs injected in an ischemic zone with the Ad-βGal construct (group 3 animals, Example 1). Blood flow (in ml/min/mg tissue) at rest (open box) and with pacing (shaded box): top left panel, in ischemic endocardial zone; top right panel, in ischemic epicardial zone; bottom left panel, in non-ischemic endocardial zone; and bottom right panel, in non-ischemic epicardial zone.

FIG. 4 is a bar graph depicting myocardial blood flow in pigs injected in an ischemic zone with the Ad-VEGF₁₆₅ construct (group 1 animals, Example 1). Blood flow (in ml/min/mg tissue) at rest (open box) and with pacing (shaded box): top left panel, in ischemic endocardial zone; top right panel, in ischemic epicardial zone; bottom left panel, in non-ischemic endocardial zone; and bottom right panel, in non-ischemic epicardial zone.

FIG. 5 is a bar graph depicting myocardial blood flow in pigs injected in a non-ischemic zone with the Ad-VEGF₁₆₅ construct (group 2 animals, Example 1). Blood flow (in ml/min/mg tissue) at rest (open box) and with pacing (shaded box): top left panel, in ischemic endocardial zone; top right panel, in ischemic epicardial zone; bottom left panel, in non-ischemic endocardial zone; and bottom right panel, in non-ischemic epicardial zone.

FIG. 6 is a bar graph depicting transmural myocardial blood flow in pigs injected with: top left panel, the Ad-βGal construct in an ischemic zone (group 3 animals, Example 1); top right panel, PBS in an ischemic zone (group 4 animals, Example 1); bottom left panel, the Ad-VEGF₁₆₅ construct in an ischemic zone (group 1 animals, Example 1); and the Ad-VEGF₁₆₅ construct in a non-ischemic zone (group 2 animals, Example 1). Blood flow (in ml/min/mg tissue) is shown at rest (open box) and with pacing (shaded box).

FIG. 7 is a bar graph depicting regional wall motion scores on dobutamine stress echocardiography. Wall motion scores are 1=normal, 2=hypokinesis, 3=akinesis, and 4=dyskinesis, pre-stress (open box), at low dose dobutamine (light shaded box), and high dose dobutamine (dark shaded box). Top left panel=the Ad-βGal construct in an ischemic zone (group 1 animals, Example 2), top right panel, the VEGF₁₆₅ construct in an ischemic zone (group 2 animals, Example 2), bottom left panel, the VEGF₁₆₅ construct in a normal zone (group 3 animals, Example 2), bottom right panel, the VEGF₁₆₅ construct in normal and ischemic zones (group 4 animals, Example 2).

FIG. 8 is a bar graph depicting myocardial blood flow in the ischemic zone of pigs injected with: Ad-βGal into an ischemic zone (top left), Ad-VEGF₁₆₅ into an ischemic zone (top right), Ad-VEGF₁₆₅ into a normal zone (bottom left) or Ad-VEGF₁₆₅ into both ischemic and normal zone (bottom right). Blood flow (in ml/min/mg tissue) at rest (open box) and with pacing (shaded box) at baseline and after treatment.

FIG. 9 is a bar graph depicting capillary density in pigs (number/mm ) injected with Ad-βGal into an ischemic zone, Ad-VEGF₁₆₅ into an ischemic zone, Ad-VEGF₁₆₅ into a normal zone, and Ad-VEGF₁₆₅ into both ischemic and normal zone.

### DETAILED DESCRIPTION OF THE INVENTION

The invention provides a method of treating ischemic heart disease by injecting a therapeutic agent into normal myocardium in an amount sufficient to induce angiogenesis, stimulate collateral blood vessel formation, improve contractile function or promote tissue regeneration. The therapeutic agent can be injected into normal myocardium adjacent to a zone of ischemic myocardium or ischemic myocardial tissue of an animal, or the therapeutic agent can be injected into multiple sites distributed throughout the normal myocardium. In a preferred embodiment the therapeutic agent is a gene therapy vector encoding at least one nucleic acid, i.e., the transgene, encoding an angiogenic factor; and expressing that factor in an amount effective to treat the ischemic heart disease or to stimulate collateral blood vessel formation, to treat or ameliorate the cardiovascular condition or to promote tissue regeneration. The invention also contemplates methods to induce angiogenesis, to increase contractile function in the heart, to increase blood flow within the heart, to stimulate collateral vessel development in the heart, to promote tissue regeneration and to treat myocardial ischemia, preferably in a human patient using the therapeutic agents of the invention.

In some embodiments, a therapeutic agent is delivered to an ischemic or diseased heart by intramyocardially delivering a therapeutically effective amount of a therapeutic agent to normal tissue in the heart. The invention also provides methods for stimulating collateral blood vessel formation in the myocardium, for inducing angiogenesis in the myocardium, and for improving contractile function of the heart by delivering an angiogenic factor or cells capable of producing an angiogenic factor to normal tissue in an ischemic or diseased heart. The angiogenic factor is preferably delivered by an adenovirus vector or an adeno-associated vector which comprises a coding sequence encoding an angiogenic factor, wherein the coding sequence is operatively linked to a promoter which can direct expression of the angiogenic factor in a cardiac cell. Preferred vectors for use with the invention include replication-defective adenoviruses, serotype 5 adenoviruses, and adenoviruses lacking the early gene region E1, the early gene region E3, or both.

The invention also provides a method for ameliorating the symptoms associated with myocardial ischemia, which comprises delivering a therapeutic agent to normal myocardial tissue in an amount sufficient to ameliorate one or more of the symptoms of ischemia. Amelioration of symptoms includes, for example, increased tolerance to exercise, decreased chest pain, and decreased shortness of breath.

The therapeutic agent can be a gene therapy vector, protein, peptide, antisense DNA or RNA, drug, cells, cells which express a therapeutic agent, whole bone marrow and or any other therapeutic agent capable of or useful to induce angiogenesis, increase contractile function in the heart, increase blood flow within the heart, stimulate collateral vessel development in the heart, treat myocardial ischemia, or promote tissue regeneration.

Any suitable gene therapy vector can be used to supply the transgene. For example, the gene therapy vector can be a replication-deficient adenovirus, a recombinant adeno-associated virus vector (rAAV), a retroviral vector, a plasmid, or any other vector useful in cardiac gene therapy. Non-limiting examples of recombinant adenoviral vectors suitable for use in the invention include the recombinant adenoviruses described in Graham et al. (Virology 163:614-617, 1988), as well as those in Graham F. et al. (Methods in Molecular Biology 7: 109-128, Murray, E., ed. Humana Press, Clifton, N.J., 1991), Curiel, et al. (Proc. Natl. Acad. Sci. USA 88:8850-8854, 1991), Miller et al. (FASEB J. 9:190-199, 1995), and Curiel (Ann. NY Acad. Sci. 886:158-171). Adenovirus vectors suitable for use with the invention also include adenoviruses of adenovirus serotype 5, and adenoviruses lacking the early gene E1 region, lacking the early gene E3 region, or lacking both. Adeno-associated vectors are described in, for example, Smith-Arica et al. (Curr. Cardiol. Rep. 3:43-49, 2001), Philips (Expert Opinion. Biol. Ther. 1:655-662, 2001), Rabinowitz, et al. (J. Virol 76:791-801). Other viral vectors suitable for use in the invention include retroviral vectors, corona virus based vectors, and vaccinia-based vectors. Plasmid and other non-viral vectors such as plasmid/liposome vectors, virus/liposome vectors, oligonucleotides, and others are described in, for example, McKay, et al. (Cariovasc. Drug. Rev. 19: 245-62,2001) and Rosenzweig (Vectors for Gene Therapy. In: Current Protocols in Human Genetics. Dracopoli, et al. eds. New York, N.Y.: John Wiley and Sons, Inc., 1999).

Gene therapy vectors useful in the present invention can be any vector with one or more transgenes (or nucleic acids of interest) inserted therein in a manner allowing expression of the transgene under control of appropriate regulatory elements such as promoters, enhancers, transcription terminators and the like. Gene therapy vectors are well known in the art and can be prepared by standard methodology known to those of ordinary skill in the art.

Further, the nucleic acid is operably linked to a control region, e.g., promoters, enhancers, termination signals and the like, to permit expression of the molecule. When more than one nucleic acid is present on the vector, each can be controlled separately by individual control regions or, any group of them, or all of them, can be controlled in an operon, i.e., with one control region driving expression of multiple genes on a single transcript.

A “transgene” or “nucleic acid of interest” or the “nucleic acid encoded in the vector” as used herein refers to any nucleotide sequence which encodes a therapeutically-effective molecule to induce angiogenesis, to stimulate collateral blood vessel formation, or to increase myocardial blood flow in ischemic areas of the heart. These transgenes can encode the proteins and angiogenic factors of the invention described herein. The transgenes can be foreign to the animal being treated, or can be genes normally found in the animal being treated, but for which altered expression, is desired. Expression can be altered by changing the amount of expression, or temporal or spatial pattern of expression.

As used herein, a “control region” or “regulatory element” refers to polyadenylation signals, upstream regulatory domains, promoters, enhancers, transcription termination sequences and the like which regulate the transcription and translation of a nucleic acid sequence.

The term “operably linked” refers to an arrangement of elements wherein the components are arranged so as to perform their usual function. Thus, control regions or regulatory elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The regulatory elements of the invention can be derived from any source, e.g., viruses, mammals, insects or even synthetic, provided that they function after injection into the heart. For example, any promoter can used to control expression of the transgene. Such promoters can be promiscuous, i.e., active in many cell types, such as the SV40 early promoter, the mouse mammary tumor virus LTR promoter, the adenovirus major late promoter (Ad MLP), a herpes simplex promoter, a CMV promoter such as the CMV immediate early promoter, or a rous sarcoma virus (RSV) promoter. Alternatively the promoter can be tissue-specific for expression in cardiac cells such as cardiomyocytes. Non-limiting examples of tissue specific promoters are known in the art (see, e.g., Lee, et al. (1992) J. Biol. Chem. 267:15875-15885; Jeyaseelan et al. (1997) Proc. Natl. Acad. Sci. USA 272:22800-22808; Condorelli, et al. (2001) Proc. Natl. Acad. Sci. USA 98:9977-9982) include the left ventricular myosin light chain-2 (MLC₂ᵥ) promoter, myosin heavy chain (MHC) promoters such as the A-MHC and P-MHC, natriuretic peptide precursor A promoter (NppA), the promoter of the cardiac adriamycin responsive protein (CARP), the promoter of the cTNC gene, and others.

Proteins that can be administered (encoded in gene therapy vectors or directly) include proteins or peptides competent to induce angiogenesis, e.g., angiogenesis factors. A protein or peptide competent to induce angiogenesis or an “angiogenesis factor” as used herein is a protein or substance that causes proliferation of new blood vessels and includes fibroblast growth factors, endothelial cell growth factors or other proteins with such biological activity. Angiogenic factors, and particular proteins known to induce angiogenesis, include but are not limited to, FGF-1, FGF-2, FGF-5, VEGF and active fragments thereof such as VEGF₁₆₅, HIF-1 PDGF-1, PDGF-2, DEL1, angiopoietins, HGF, MCP-1, eNOS and INOS. Other angiogenic factors suitable for use in the invention are growth factors, including endothelial growth factors, vascular smooth muscle growth factors, and FGF-1, FGF-2, FGF-5, PDGF-1, and PDGF-2. The abbreviations are as follows: FGF, fibroblast growth factor; VEGF, vascular endothelial growth factor; HIF, hypoxia inducible factor; PDGF, platelet-derived growth factor; DEL, developmental embryonic locus: HGF, hepatocyte growth factor; MCP, monocyte chemoattractant protein; eNOS, endothelial nitrous oxide synthase; and iNOS, inducible nitrate oxide synthase.

Other proteins or transgenes are also suitable for use in the invention, for example factors involved in myocardial preservation or reperfusion injury, such as heme oxygenase, hkis, AKT, PR39, and βarkCT, can be used in the methods of the invention. Tissue regeneration factors, including, but not limited to, ligands for progenitor or stem cells, such as c-kit ligand, CD34 ligand, and other factors are also suitable for use in the methods of the invention.

Cells that can be administered by the present method include, but are not limited to, endothelial progenitor cells (angioblasts), cardiac myoblasts, mononuclear cells, bone marrow stromal cells and stem cells. “Stem cells” as used herein refers to mononuclear cells from placental or umbilical cord blood. The cells described herein can be administered as primary cells, i.e., without transformation or other ex vivo manipulation. Alternatively, any of these cells, or other appropriate cell types, can be manipulated or expanded ex vivo, or genetically engineered ex vivo or selected to produce an angiogenic factor using methods known in the art. Typically, cells are engineered to produce an angiogenic factor are engineered to secrete the desired angiogenic factor. Additionally, filtered whole bone marrow is know to be angiogenic and such a preparation can be administered in accordance with the invention.

The therapeutic agents described herein can be administered singly or in combination. In one non-limiting example, a therapeutic agent according to the invention may comprise a viral vector delivered in combination with angiogenic cells. In another non-limiting example, a therapeutic agent according to the invention may comprise a viral vector delivered in combination with an angiogenic protein. The therapeutic agents can also be delivered in combination with other active agents, such as anti-apoptotic agents.

Pharmaceutical formulations of the therapeutic agents of the invention are prepared for storage by mixing those entities having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the polypeptide variant, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylenevinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

Those of skill in the art can readily determine the amounts of the therapeutic agents to be included in any pharmaceutical composition and the appropriate dosages for the contemplated use.

The method of the present invention can be used with any animal, including but not limited to, mammals such as rodents, dogs, cats, cattle, primates and humans. Preferably the method is used for gene therapy to treat human ischemic cardiac conditions or diseases.

The amount of the therapeutic agent injected into the animal is proportional to the body weight of the animal and also depends on the selected agent. Those of skill in the art can readily determine the appropriate dosage for the selected agent. By way of example, when the agent is a gene therapy vector such as a replication-defective adenovirus, the dosage can range from about 10⁶ to about 10¹² plaque-forming units (pfu), and is preferably between about 10⁸ to about 10¹⁰ pfu. For stable and efficient transduction using rAAV, the dosage can be from about 1×10⁵ IU (infectious units) of AAV per gram body weight to about 1×10⁹ IU AAV per gram body weight, and preferably from about 1×10⁶ IU AAV per gram body weight to about 1×10⁷ IU AAV per gram body weight. When the agent is a protein, the dosage can range from as little as about 1 picograms to several hundred micrograms, but in any event can be readily determined by those of skill in the art.

Methods for measuring cardiac function are well known in the art. See, e.g., Simons et al. (2000) Circulation 102:e732-e86, “Clinical Trails in Coronary Angiogenesis: Issues, Problems and Consensus.” For example, blood flow to ischemic myocardium can be measured using various non-invasive imaging techniques, including single photon emission computed tomography (SPECT), position emission tomography (PET), magnetic resonance imaging (MRI), and injection of fluorescent microspheres. Coronary angiography can be used to measure disease progression and to document the appearance of new vessels. Echocardiography can be used to assess cardiac wall motion at rest and under stress, such as dobutamine-induced stress. Exercise tolerance testing such as treadmill testing can provide another means for assessing cardiac function.

Delivery to myocardium can be accomplished using a catheter (e.g. infusion catheter, diagnostic catheter, etc) stiletto catheter, needle or needles, needle-free injector, balloon catheter, channeling device, or other appropriate medical device for introduction into the myocardium. In a preferred delivery method, an endocardial injection catheter, such as a Stiletto catheter (Boston Scientific, Natick, Mass.) is used to deliver the therapeutic agent without requiring open chest surgery Catheter injections can be guided by fluoroscopy, echocardiography, MRI, or electromechanical mapping. The catheter is used to deliver the therapeutic agent to non-ischemic tissue in the myocardium by transendocardial injection. Appropriate devices and methods for catheter injection are described in U.S. Pat. No. 6,238,406. Alternatively, a transepicardial surgical approach may be necessary for delivery to myocardium, either via open chest or via thoracoscopy.

Throughout this application, various publications, patents, and patent applications have been referred to. The teachings and disclosures of these publications, patents, and patent applications in their entireties are hereby incorporated by reference into this application to more fully describe the state of the art to which the present invention pertains.

It is to be understood and expected that variations in the principles of invention herein disclosed in an exemplary embodiment may be made by one skilled in the art and it is intended that such modifications, changes, and substitutions are to be included within the scope of the present invention.

### EXAMPLE 1

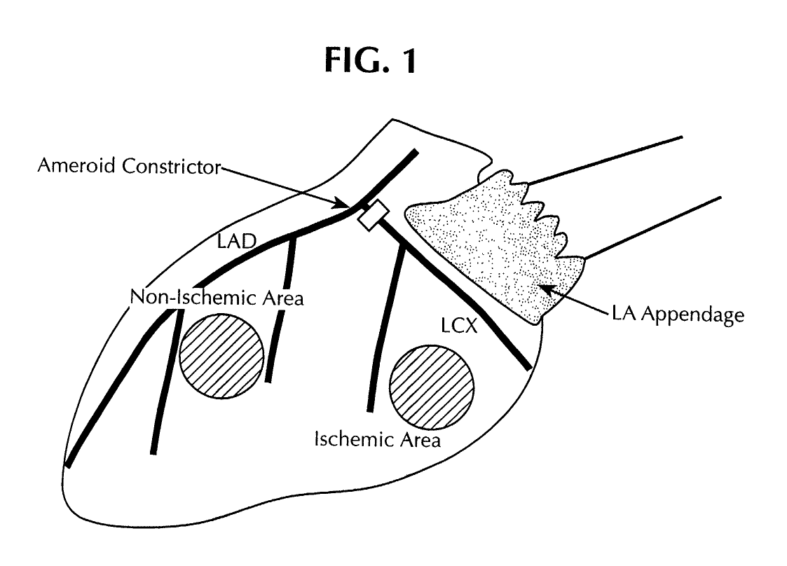

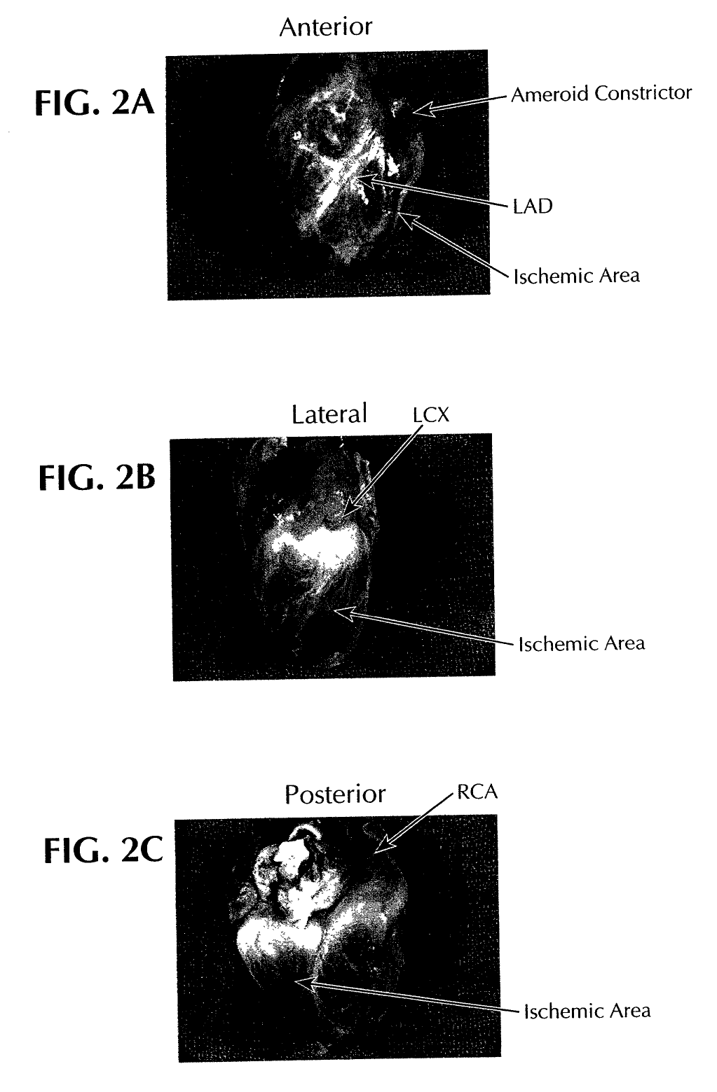

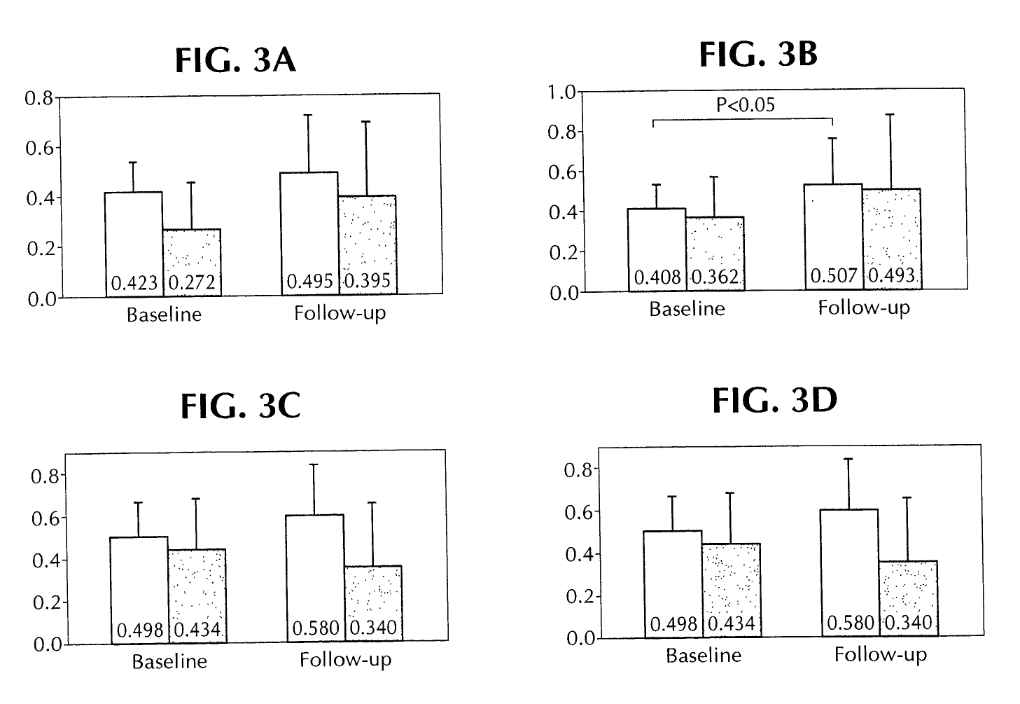

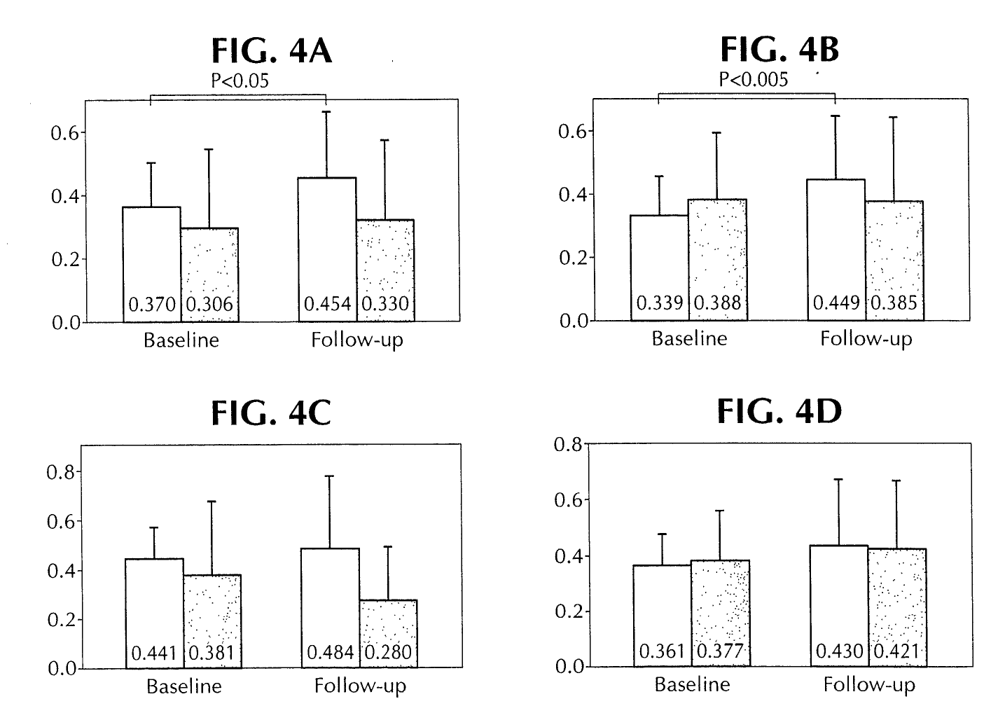

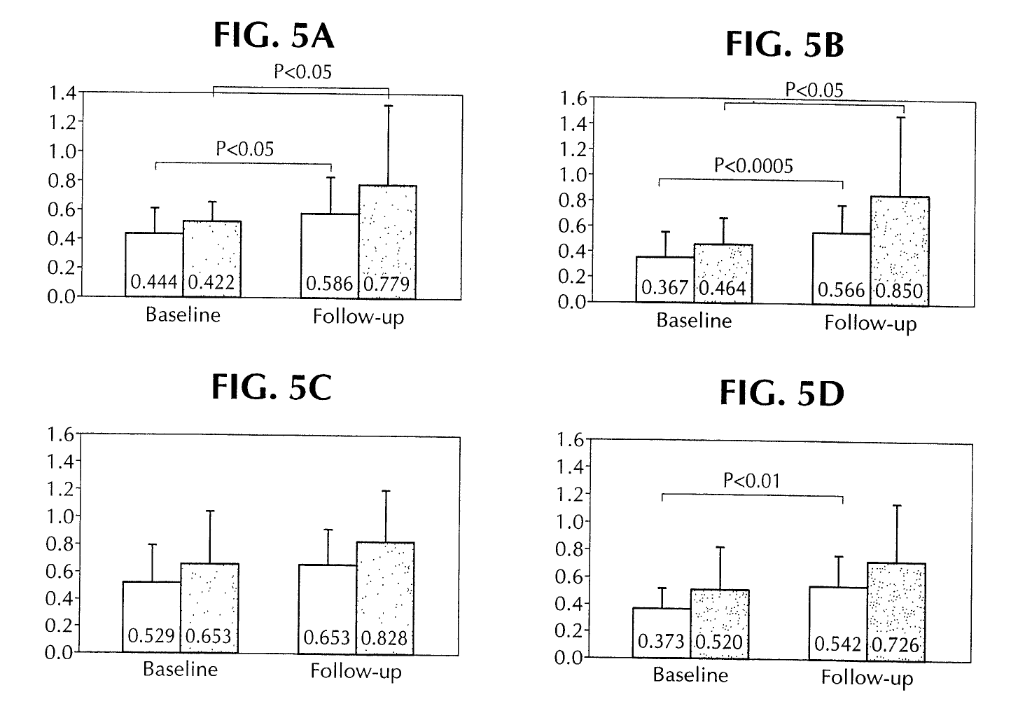

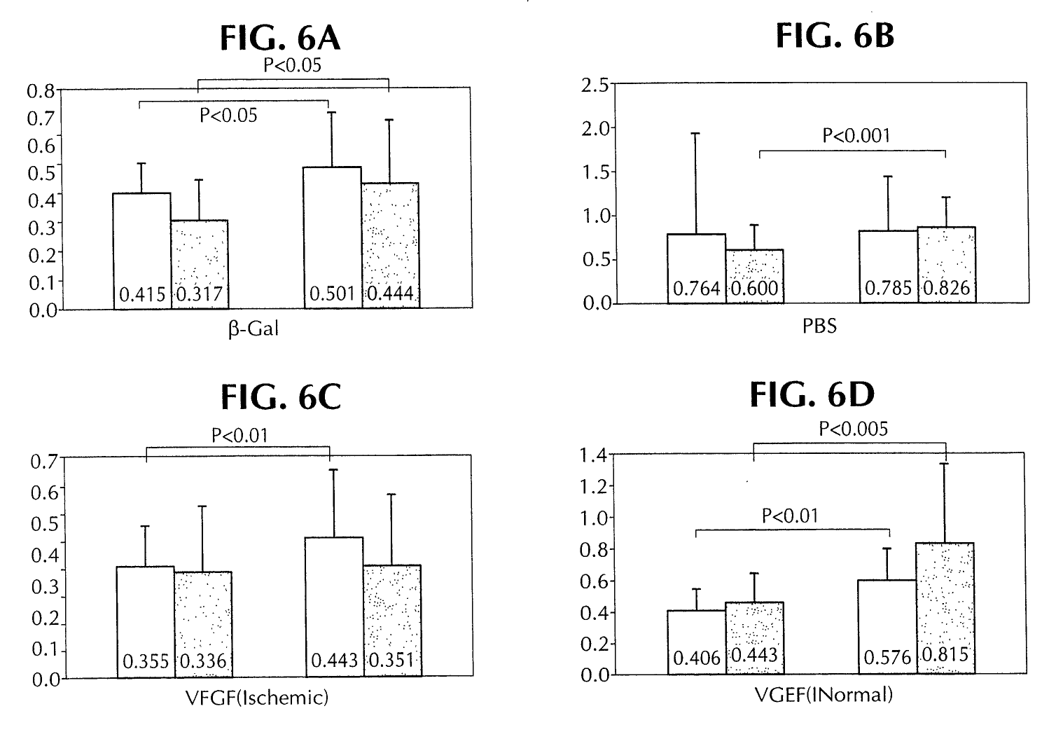

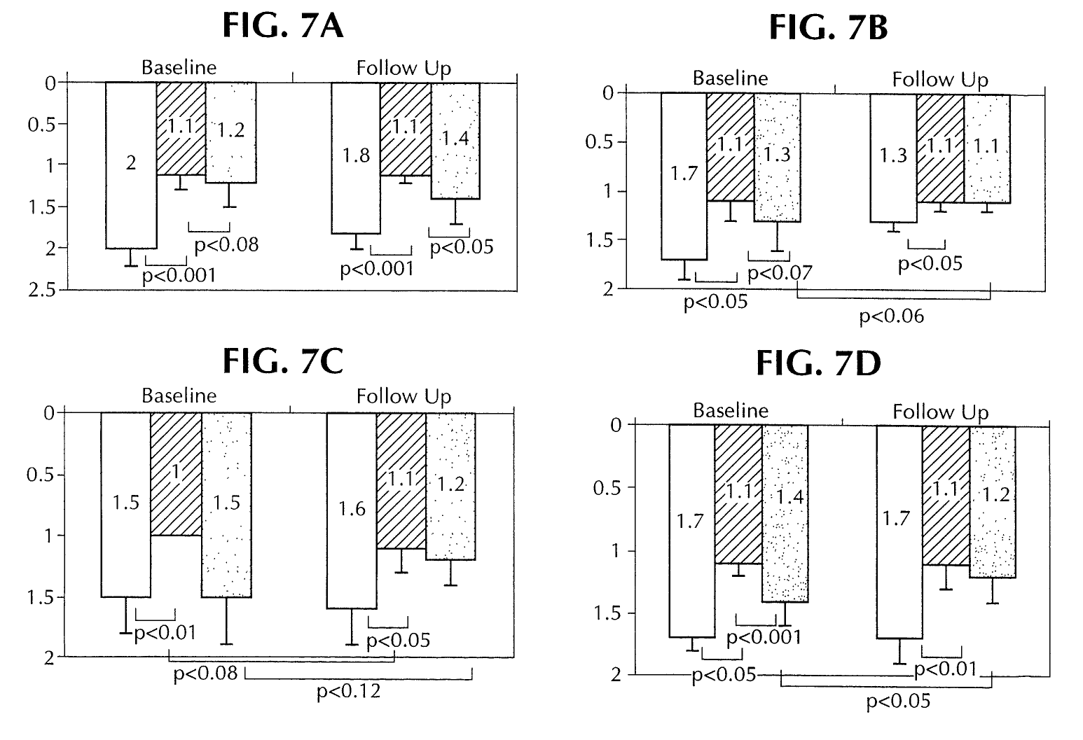

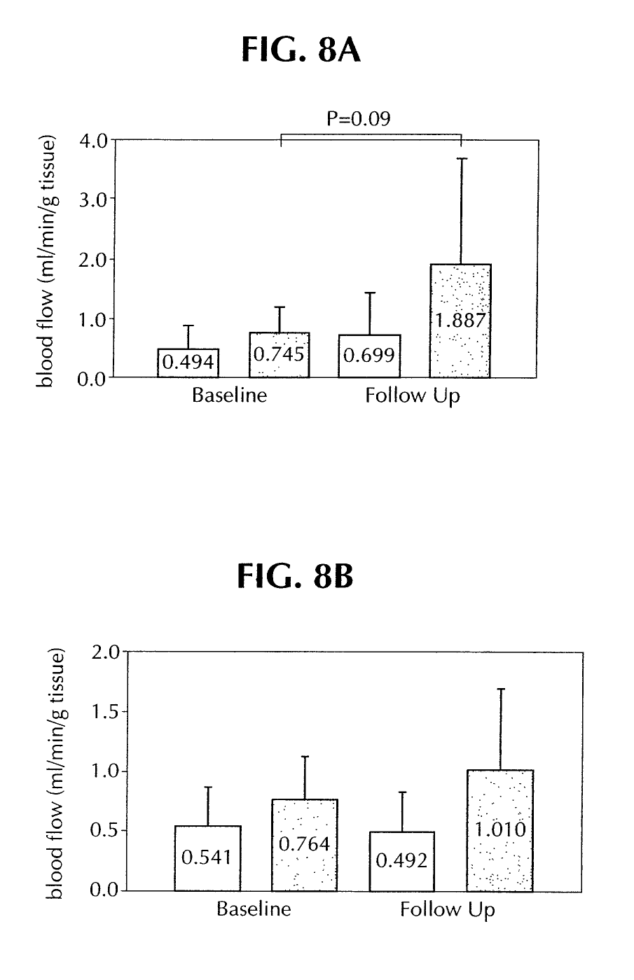

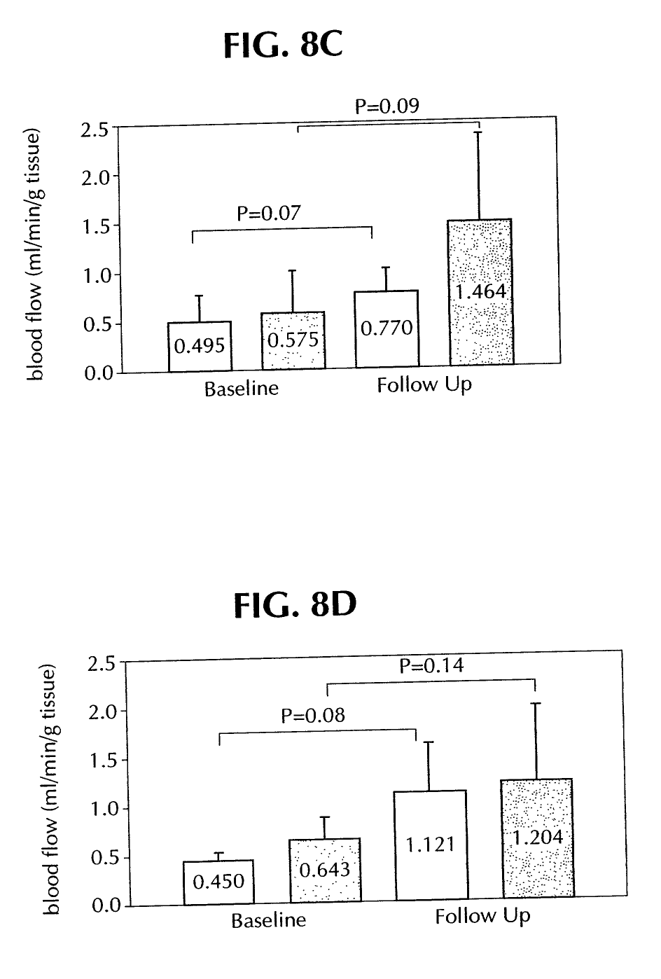

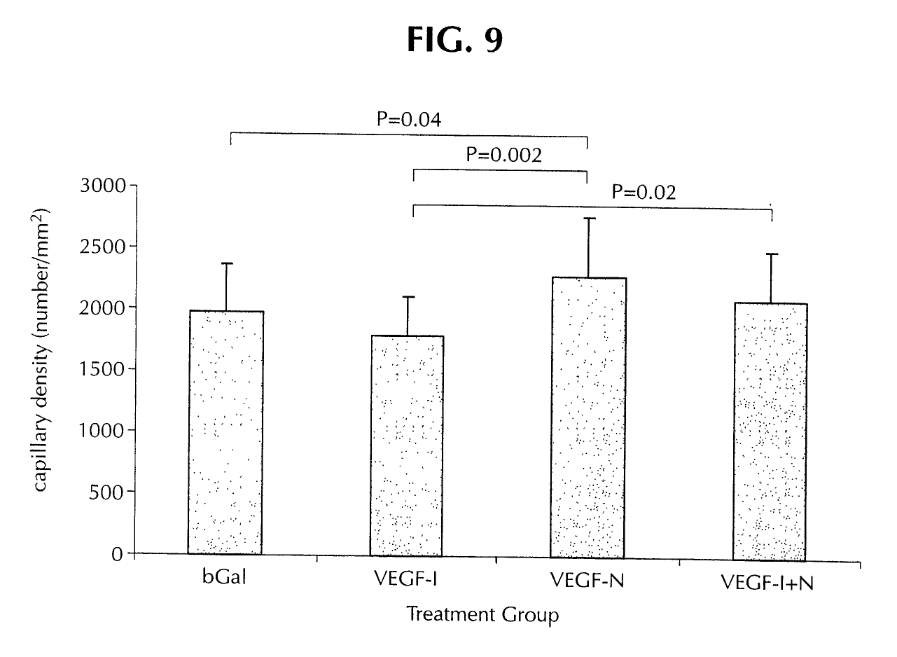

Induction of Chronic Myocardial Ischemia: Juvenile cross bred pigs (˜20-25 kg) underwent left lateral thoracotomy. An ameroid constrictor was placed around the proximal LCX just distal to the main stem of the left coronary artery using an ameroid constrictor matching the size of the vessel, typically 1.75, 2.00 or 2.25 mm inner diameter (ID). FIG. 1 illustrates placement of the ameroid constrictor.

Assessment of Cardiac Function and Myocardial Injections: Baseline measurements of cardiac function were obtained four weeks after placement of the ameroid constrictor. The measurements included coronary angiography, dobutamine stress echocardiography, blood flow measurements by injection of microspheres at rest and at atrial pacing of 180 beats per minute (bpm).

After baseline measurements were completed, vectors or saline were introduced into the heart in the indicated zones by intramyocardial injection as described in Kornowski et al. (2000) J. Am. Coll. Card. 35:1031-1039. This method allows direct injection into normal or ischemic myocardium during open-heart surgery with a magnetic guidance catheter-based navigational system. The injections consisted of 10 injections of 20 μL of 5×10⁹ pfu/mL of Ad-VEGF₁₆₅ or Ad-βGal or 10 injections of 20 μL of phosphate-buffered saline (PBS).

Four weeks after the injections, i.e., eight weeks after implantation of the ameroid constrictor, the baseline measurements were repeated. Additionally, ischemic and adjacent normal areas were harvested post-mortem for regional myocardial blood flow measurement, histopathologic analysis and morphometric analysis.

Treatment Groups: The animals were divided into four groups and received injection of (1) Ad-VEGF₁₆₅ into the ischemic zone (n=9); (2) Ad-VEGF₁₆₅ into the normal zone (n=8); (3) Ad-βGal into the ischemic zone (n=8); or (4) PBS into the ischemic zone (n=7).

Results: The blood flow data indicate that when injections are targeted to the ischemic zone, modest improvements in perfusion occur at rest. However, when injections are made in to the normal zone of the myocardium, significant improvements are observed in blood perfusion at both rest and stress. Further more, transmural blood flow reaches a much higher level of 0.815 (normal zone injections) versus 0.351 (ischemic zone injections) under stress.

### EXAMPLE 2

Induction of Chronic Myocardial Ischemia: Ameroid constrictors were placed around the proximal LCX of juvenile pigs via left lateral thoracotomy as described in Example 1.

Assessment of Cardiac Function and Myocardial Injections: Baseline measurements of cardiac function were obtained four weeks after placement of the ameroid constrictor. The measurements included coronary angiography, dobutamine stress echocardiography, blood flow measurements by injection of fluorescent microspheres at rest and at atrial pacing of 180 beats per minute (bpm).

After baseline measurements were completed, vectors or saline were introduced into the heart by Stiletto injection catheter. Each animal received 10 injections, each 20 μL, of 5×10⁹ pfu/mL of Ad-VEGF₁₆₅ or Ad-βGal.

Four weeks after the injections, i.e., eight weeks after implantation of the ameroid constrictor, the baseline measurements were repeated. Additionally, ischemic and adjacent normal areas were harvested post-mortem for regional myocardial blood flow measurement, histopathologic analysis, and morphometric analysis.

Treatment Groups: The animals were divided into four groups and received injection of (1) Ad- PGal into the ischemic zone (n=7); (2) Ad-VEGF₁₆₅ into the ischemic zone (n=7); (3) AdVEGF₁₆₅ into the normal tissue adjacent to the ischemic zone (n=7); and (4) AdVEGF₁₆₅ throughout the left ventricular free wall in both normal and ischemic tissue (n=8).

Results: Under resting conditions, animals that received injections of Ad-βGal into the ischemic zone did not show significant improvement in blood flow at rest, but did show improvement in blood flow with pacing. Trends toward improvement in blood flow were not seen in animals that received injections of AdVEGF₁₆₅ into the ischemic region. Animals that received injections of Ad-VEGF₁₆₅ into the normal zone showed trends toward improvement both at rest and with pacing. Animals that received injections throughout the left ventricular free wall in both ischemic and normal zone also showed trends toward improvement both at rest and with pacing.

Dobutamine stress echocardiography indicated trends toward improvement in wall motion in all animals that received the Ad-VEGF₁₆₅ construct. In contrast, animals that received the Ad-βGal construct showed decrements in wall motion.

Animals that received injections of Ad-VEGF₁₆₅ in the ischemic zone had lower capillary density than animals that received Ad-βGal in the ischemic zone. Animals that received injections of Ad-VEGF₁₆₅ in the normal zone had higher capillary density than animals that received injections of Ad-βGal in the ischemic zone, and animals that received injections of Ad-VEGF₁₆₅ in both the normal and ischemic zones had capillary density similar those that received injections of Ad-βGal in the ischemic zone.

1. A method for delivering a therapeutic agent to an ischemic or diseased heart which comprises intramyocardially delivering a thereapeutically-effective amount of said therapeutic agent to normal tissue in said heart.

2. The method of claim 1, wherein the therapeutic agent is delivered to normal tissue adjacent to ischemic tissue or diseased tissue.

3. The method of claim 1 or 2, wherein the therapeutic agent is injected at multiple sites in the normal tissue.

4. The method of claims 1, wherein the therapeutic agent is a protein.

5. The method of claim 4, wherein the protein is selected from the group consisting of FGF-1, FGF-2, FGF-5, VEGF, VEGF165, HIF-1, PDGF-1, PDGF-2, DEL1, angiopoietin, HGF, MCP-1, eNOS, and INOS.

6. The method of claim 1, wherein the therapeutic agent comprises a nucleic acid.

7. The method of claim 6, wherein the nucleic acid encodes a protein selected from the group consisting of FGF-1, FGF-2, FGF-5, VEGF, VEGF165, HIF-1, PDGF-1, PDGF-2, DEL1, angiopoietin, HGF, MCP-1, eNOS, and INOS.

8. The method of claim 6, wherein the nucleic acid encodes an antisense molecule.

9. The method of claim 6, wherein the nucleic acid is delivered as RNA, DNA, plasmid or a viral vector.

10. The method of claim 9, wherein the viral vector is an adenovirus vector or an adeno-associated virus vector.

11. The method of claim 10, wherein said RNA, DNA, plasmid or a viral vector is in a liposome.

12. The method of claim 1, wherein the therapeutic agent comprises cells.

13. The method of claim 12, wherein said cells are endothelial progenitor cells, mononuclear cells, bone marrow stromal cells, stem cells, cardiac myoblasts, are the cells of whole filtered bone marrow, or any combination of cells.

14. The method of claim 12 or 13, wherein said cells have been genetically engineered ex vivo.

15. A method to stimulate collateral blood vessel formation in the myocardium which comprises intramyocardially delivering an angiogenic factor, or cells capable of producing an angiogenic factor, to normal tissue in an ischemic heart of a subject in an amount sufficient to stimulate collateral blood vessel formation.

16. The method of claim 15, wherein said angiogenic factor is an adenovirus vector or an adeno-associated virus vector comprising a coding sequence encoding said angiogenic factor, said sequence operatively linked to a promoter effective to induce expression in a cardiac muscle cell, and wherein sufficient expression of said angiogenic factor occurs to stimulate collateral blood vessel formation.

17. A method to induce angiogenesis in the myocardium which comprises intramyocardially delivering an angiogenic factor, or cells capable of producing an angiogenic factor, to normal tissue in an ischemic heart of a subject in an amount sufficient to induce angiogenesis.

18. The method of claim 17, wherein said angiogenic factor is an adenovirus vector or an adeno-associated virus vector comprising a coding sequence encoding said angiogenic factor, said sequence operatively linked to a promoter effective to induce expression in a cardiac muscle cell, and wherein sufficient expression of said angiogenic factor occurs to induce angiogenesis.

19. A method to improve contractile function of in an ischemic heart which comprises intramyocardially delivering an angiogenic factor, or cells capable of producing an angiogenic factor, to normal tissue in an ischemic heart of a subject in an amount sufficient to improve contractile function of said heart.

20. The method of claim 19, wherein said angiogenic factor is an adenovirus vector or an adeno-associated virus vector comprising a coding sequence encoding said angiogenic factor, said sequence operatively linked to a promoter effective to induce expression in a cardiac muscle cell, and wherein sufficient expression of said angiogenic factor occurs to improve contractile function of said heart.

21. A method to promote tissue regeneration in an ischemic or diseased heart which comprises intramyocardially delivering a therapeutic agent to normal tissue in an ischemic heart of a subject in an amount sufficient to stimulate tissue regeneration in said heart.

22. The method of claim 21, wherein said therapeutic agent is an adenovirus vector or an adeno-associated virus vector comprising a coding sequence encoding a ligand for a progenitor or stem cell, said sequence operatively linked to a promoter effective to induce expression in a cardiac muscle cell, and wherein sufficient expression of said ligand occurs to stimulate tissue regeneration in said heart.

23. The method of any one of claims 15-20, wherein the angiogenic factor or cells are delivered to normal tissue adjacent to ischemic tissue.

24. The method of any one of claims 15-20 wherein the angiogenic factor or cells are injected at multiple sites in the normal tissue.

25. The method of any one of claims 15-20, wherein said angiogenic factor is selected from the group consisting of FGF-1, FGF-2, FGF-5, VEGF, VEGF₁₆₅, HIF-1, PDGF-1, PDGF-2, DEL1, angiopoietin, HGF, MCP-1, eNOS, INOS, or a combination thereof.

26. The method of any one of claims 15-20 wherein said angiogenic factor is a growth factor.

27. The method of claim 26, wherein said growth factor is FGF-5, acidic FGF, basic FGF, PDGF1, PDGF2, VEGF an endothelial growth factor or a vascular smooth muscle growth factor.

28. The method of any one of claims 15, 17, or 19, wherein said angiogenic factor is a protein or encoded by a nucleic acid with the coding sequence for said protein operatively linked to a promoter effective to induce expression of said protein in a cardiac muscle cell.

29. The method of claim 28, wherein said promoter is tissue-specific.

30. The method of claim 29, wherein the tissue-specific promoter is selected from the group consisting of the promoters of cTNC, MHCα, MHCβ, MLC₂ᵥ, NppA, and CARP.

31. The method of any one of claims 16, 18, 20, or 22, wherein said promoter is tissue-specific.

32. The method of claim 31, wherein the tissue-specific promoter is selected from the group consisting of the promoters of MHCα, MHCβ, MLC₂ᵥ, NppA, and CARP.

33. The method of any one of claims 16, 18, 20 or 22, wherein said adenovirus is replication-defective.

34. The method of any one of claims 16, 18, 20 or 22, wherein said adenovirus is adenovirus serotype 5.

35. The method of any one of claims 16, 18, 20 or 22, wherein said adenovirus lacks the early gene region E1, the early gene region E3, or both.

36. The method of claim 21, wherein the therapeutic agent is a protein, nucleic acid, or drug which promotes tissue regeneration.

37. The method of claim 21, wherein the therapeutic agent is the CD34 ligand or the c-kit ligand.

38. A method for treating myocardial ischemia which comprises delivering a therapeutic agent to normal myocardial tissue in an amount sufficient to ameliorate the symptoms of ischemia.

39. The method of claim 38, wherein the therapeutic agent is delivered to normal tissue adjacent to ischemic tissue.

40. The method of claim 38 or 39, wherein the therapeutic agent is injected at multiple sites in the normal tissue.

41. The method of claim 38, wherein the therapeutic agent is a protein.

42. The method of claim 41, wherein the protein is selected from the group consisting of FGF-1, FGF-2, FGF-5, VEGF, VEGF165, HIF-1, PDGF-1, PDGF-2, DEL1, angiopoietin, HGF, MCP-1, eNOS, and INOS.

43. The method of claim 38, wherein the therapeutic agent comprises a nucleic acid.

44. The method of claim 43, wherein the nucleic acid encodes a protein selected from the group consisting of FGF-1, FGF-2, FGF-5, VEGF, VEGF165, HIF-1, PDGF-1, PDGF-2, DEL1, angiopoietin, HGF, MCP-1, eNOS, and INOS.

45. The method of claim 43, wherein the nucleic acid encodes an antisense molecule.

46. The method of claim 43, wherein the nucleic acid is delivered as RNA, DNA, plasmid or a viral vector.

47. The method of claim 46, wherein the viral vector is an adenovirus vector or an adeno-associated virus vector.

48. The method of claim 46, wherein said RNA, DNA, plasmid or a viral vector is in a liposome.

49. The method of claim 38, wherein the therapeutic agent comprises cells.

50. The method of claim 49, wherein said cells are endothelial progenitor cells, mononuclear cells, bone marrow stromal cells, stem cells, cardiac myoblasts, are the cells of whole filtered bone marrow, or any combination of cells.

51. The method of claim 49 or 50, wherein said cells have been genetically engineered ex vivo.

52. The method of claim 38, wherein an amelioration of symptoms of ischemia is indicated by increased transmural blood flow in the myocardium at rest or under stress conditions, by collateral blood vessel formation, by improved contractile function or by regeneration of myocardial tissue.

53. The method of claim 38, wherein an amelioration of one or more symptoms of ischemia is indicated by reduced chest pain or reduced shortness of breath.

54. The method of any one of claims 1, 2, 4, 6, 8, 12, 15-22, 38, 39, 41, 43, 45, 49 or 52, wherein said delivery is by a catheter, a stiletto catheter, a needle, a needle-free injector, or a channeling device.
